(19) United States
(12) Patent Application Publication
(10) Pub. No.: US 2019/0374198 A1
(43) Pub. Date: Dec. 12, 2019

(54) METHOD AND SYSTEM FOR COVERING AN ULTRASOUND PROBE

(71) Applicant: General Electric Company, Schenectady, NY (US)

(72) Inventors: Paul Lawrence Mullen, Waukesha, WI (US); Robert Anderson, Oshkosh, WI (US); Emily Elizabeth Siira, Waukesha, WI (US); Robert Meurer, Waukesha, WI (US)

(21) Appl. No.: 16/002,907

(22) Filed: Jun. 7, 2018

(51) Int. Cl. A61B 8/00; A61B 50/30

(52) U.S. Cl. CPC A61B 8/4422; A61B 8/4272; A61B 2050/314; A61B 2562/242; A61B 50/30

(57) ABSTRACT

A system and method for covering an ultrasound probe by a probe cover application device is provided. The system includes a frame, attachment device, and probe cover sheathing. The frame includes a base having a top side, bottom side, inner edge, and outer edge. The frame includes an extension extending from the top side of the base and substantially perpendicular to the base. The extension includes an inside surface and outside surface. The inner edge of the base and the inside surface of the extension form an opening through the frame. The attachment device is mounted to or integrated with the bottom side of the base of the frame. The probe cover sheathing is bunched up and/or rolled up and positioned against and surrounding the outside surface of the extension. A first closed end of the probe cover sheathing extends over the opening in the extension.

### FIELD

Certain embodiments of the disclosure relate to ultrasound probe cover application systems and methods. More specifically, certain embodiments of the disclosure relate to a method and system for covering an ultrasound probe by securing a probe cover application device to a probe holder that is holding the ultrasound probe and pulling the covered ultrasound probe through the secured probe cover application device.

### BACKGROUND

Ultrasound probe covers are often used to maintain sterile environments in order to prevent infections during medical procedures and/or medical imaging examinations. For example, ultrasound probe covers may be employed on ultrasound probes providing surgical guidance, such as during a needle biopsy, while inserting a catheter in a blood vessel, and when injecting antiesthetic into a muscle or tendon. As another example, ultrasound probe covers may be used on intracavitary ultrasound probes, such as transvaginal, transrectal, and transesophageal probes. Additionally, ultrasound probe covers may be utilized in high risk situations, such as if a patient has an open wound or infectious disease.

Conventional ultrasound probe covers are typically applied using one or two people using a total of two or three hands. For example, a non-sterile first person that is holding the ultrasound probe with one hand may insert the probe into the cover being held with two hands by a sterile second person that pulls or unrolls the cover over the inserted probe. As another example, one sterile person may hold a rolled or bunched up cover and place it over the ultrasound probe without directly touching the probe. Then, while holding the probe with one hand through the cover and without directly touching the probe, the person may attempt to unroll the cover over the probe using the other hand. Both of the above-mentioned conventional examples for applying a sterile ultrasound probe cover to a non-sterile ultrasound probe are difficult and otherwise awkward to accomplish.

Further limitations and disadvantages of conventional and traditional approaches will become apparent to one of skill in the art, through comparison of such systems with some aspects of the present disclosure as set forth in the remainder of the present application with reference to the drawings.

### BRIEF SUMMARY

A system and/or method is provided for covering an ultrasound probe by a probe cover application device, substantially as shown in and/or described in connection with at least one of the figures, as set forth more completely in the claims.

These and other advantages, aspects and novel features of the present disclosure, as well as details of an illustrated embodiment thereof, will be more fully understood from the following description and drawings.

### BRIEF DESCRIPTION OF SEVERAL VIEWS OF THE DRAWINGS

FIG. 1 illustrates an exemplary packaged probe cover application system being opened, in accordance with various embodiments.

FIG. 2 illustrates an exemplary probe cover application system having accessories and a probe cover application device being removed from backing, in accordance with various embodiments.

FIG. 3 illustrates an exemplary probe cover application device being situated over an ultrasound probe set in a probe holder, in accordance with an embodiment of the disclosure.

FIG. 4 illustrates an exemplary probe cover application device moving onto an ultrasound probe set in a probe holder, in accordance with an embodiment of the disclosure.

FIG. 5 illustrates an exemplary probe cover application device being secured to the probe holder, in accordance with an embodiment of the disclosure.

FIG. 6 illustrates exemplary probe cover sheathing of an exemplary probe cover application device being applied to an ultrasound probe, in accordance with an embodiment of the disclosure.

FIG. 7 illustrates exemplary probe cover sheathing of an exemplary probe cover application device being secured to the ultrasound probe cord, in accordance with an embodiment of the disclosure.

FIG. 8 is a flow chart illustrating exemplary steps that may be utilized for covering an ultrasound probe with an exemplary probe cover application device, in accordance with an embodiment of the disclosure.

### DETAILED DESCRIPTION

Certain embodiments of the disclosure may be found in a method 200 and system for covering an ultrasound probe 10 by a probe cover application device 120. More specifically, aspects of the present disclosure allow a single user using one hand to cover the ultrasound probe 10 by securing a probe cover application device 120 to a probe holder 20 that is holding the ultrasound probe 10 and pulling the covered ultrasound probe 10 through the secured probe cover application device 120.

The foregoing summary, as well as the following detailed description of certain embodiments will be better understood when read in conjunction with the appended drawings. It should be understood that the various embodiments are not limited to the arrangements and instrumentality shown in the drawings. It should also be understood that the embodiments may be combined, or that other embodiments may be utilized and that structural changes may be made without departing from the scope of the various embodiments of the present disclosure. The following detailed description is, therefore, not to be taken in a limiting sense, and the scope of the present disclosure is defined by the appended claims and their equivalents.

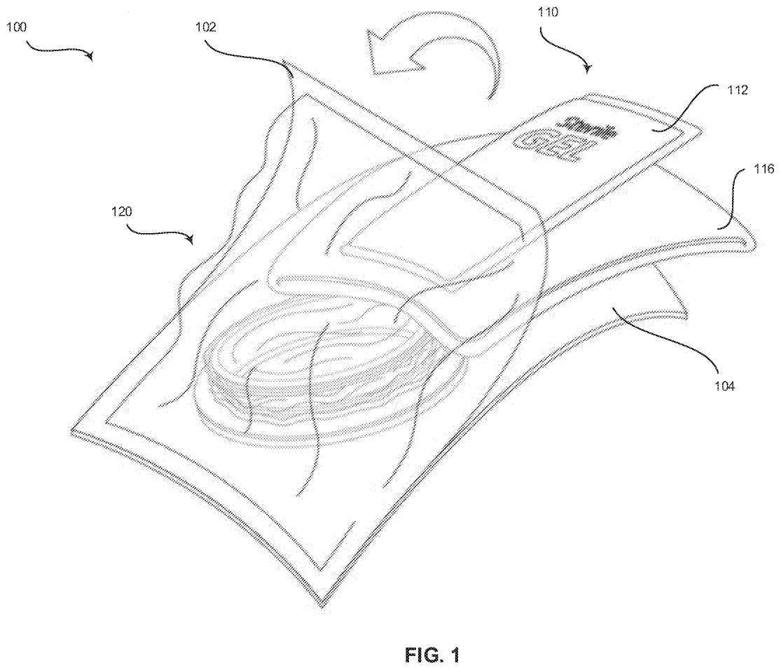

FIG. 1 illustrates an exemplary packaged probe cover application system 100 being opened, in accordance with various embodiments. Referring to FIG. 1, the packaged probe cover application system 100 may include a package contents 110, 120 disposed in packaging 102, 104. The packaging may include, for example, a package cover 102 detachably coupled to a backing 104. The detachable coupling may be an adhesive, perforations, or any suitable device for removably attaching the package cover 102 to the backing 104 such that the package cover 102 can be torn off or otherwise removed from the backing 104 to access the package contents 110, 120. The interior of the packing 102, 104 provides a sterile environment for storing the package contents 110, 120 prior to use. The package contents 110, 120 may include a probe cover application device 120 and accessories 110. The accessories 110 may include, for example, a sterile gel packet 112, tissue paper 116, and/or any suitable accessories for the probe cover application device 120. As an example, the sterile gel packet 112 may be opened to access gel 114 that can be applied to a transducer head 12 of an ultrasound probe 10 prior to covering the ultrasound probe 10 with the probe cover application device 120. The tissue paper 116 may be used, for example, to clean up any misapplied gel 114.

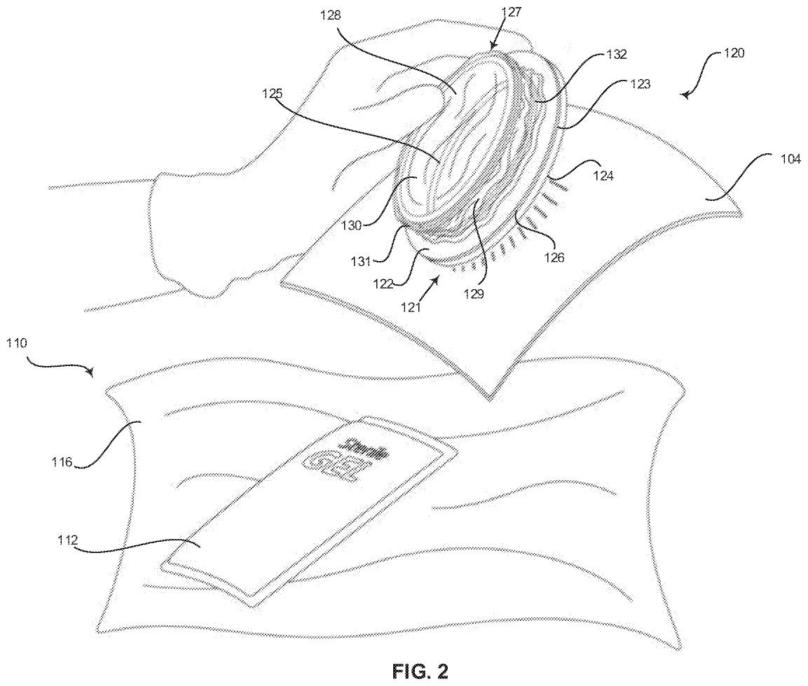

FIG. 2 illustrates an exemplary probe cover application system 110, 120 having accessories 110 and a probe cover application device 120 being removed from backing 104, in accordance with various embodiments. Referring to FIG. 2, the probe cover application system 110, 120 may include a probe cover application device 120 and accessories 110. The accessories may include, for example, tissue paper 116 and a sterile gel packet 112 having gel 114 disposed therein. In various embodiments, the probe cover application device 120 may be removably attached to a backing 104. The backing 104 may be the same backing 104 of the packaging 102, 104 as discussed above with respect to FIG. 1. Alternatively, the backing 104 may be a separate backing 104 or may be omitted.

The probe cover application device 120 may include a base 121, an extension 127, an attachment device 124, and probe cover sheathing 130. The base 121 may have a top side 122, bottom side 123, inner edge 125, and outer edge 126. The inner edge 125 may define an opening in the base 121. In various embodiments, the attachment device 124 may be attached to and/or integrated with the bottom side 123 of the base 121. The attachment device 124 may be an adhesive configured to stick to a top surface 22 of a probe holder 20, detents or protrusions configured to mate with corresponding protrusions or detents on the top surface 22 of the probe holder 20, a male or female snap portion configured to mate with a corresponding female or male snap portion on the top surface 22 of the probe holder 20, hook and loop fasteners configured to mate with corresponding hook and loop fasteners on the top surface 22 of the probe holder 20, threads configured to mate with corresponding threads on the probe holder 20, or any suitable attachment device. In an exemplary embodiment, an adhesive attachment device 124 may be peeled from the backing 104 as shown in FIG. 2 prior to application over an ultrasound probe 10 and onto a top surface 22 of a probe holder 20 as described below.

The extension 127 may include an inside surface 128 and outside surface 129. The extension 127 may be integrated or attached to the base 121 at the top side 122 of the base 121 adjacent the inner edge 125 of the base 121 and extend outward from the base 121. The extension 127 may be substantially perpendicular (defined as 90-110 degree angle between outside surface 129 of extension 127 and top side 122 of the base 121) to the base 121. The inside surface 128 of the extension 127 may form an opening in the extension 127. The opening in the extension 127 and the opening in the base 121 may form an opening through the probe cover application device 120. In certain embodiments, the inside surface 128 of the extension 127 may align with the inner edge 125 of the base 121. In various embodiments, the base 121 and extension 127 may be formed by injection molding or any suitable manufacturing process. The base 121 and extension 127 may be plastic, such as nylon, or any suitable material.

The probe cover sheathing 130 may be latex, nitrile rubber, or any suitable material. The probe cover sheathing 130 may have a first closed end and a second open end. The probe cover sheathing 130 may wrap around the outside surface 129 of the extension 127 adjacent the top side 122 of the base 121 and the first closed end may cover the opening formed by the extension 127. The portion of the probe cover sheathing 130 wrapped around the outside surface 129 of the extension 127 may be bunched up or rolled. For example, the length of the unraveled probe cover sheathing 130 is substantially greater than the height of the extension 127 so the probe cover sheathing 130 may be bunched up or rolled to store the probe cover sheathing 130 on the extension 127 prior to and during deployment onto an ultrasound probe 10. In various embodiments, one or more securement bands 131, 132 may wrap around the extension 127 over the probe cover sheathing 130. The securement band(s) 131, 132 may be strategically positioned on the probe cover sheathing 130 such that the band(s) 131, 132 deploy with the probe cover sheathing 130 to secure the probe cover sheathing 130 to the ultrasound probe 10. As an example and as described in more detail below, a first securement band 131 may be positioned near a first closed end of the sheathing 130 (defined as 1-6 inches from a closed end of the sheathing 130) to be deployed first to secure the first end of the sheathing 130 to a probe body 14 and a second securement band 132 may be positioned near a second open end of the sheathing 130 (defined as 1-6 inches from the open end of the sheathing 130) to be deployed second to secure the second open end of the sheathing 130 to a probe cord 16.

The probe cover application device 120 and accessories 110 illustrated in FIG. 2 share various characteristics with the probe cover application device 120 and accessories 110 illustrated in FIG. 1 as described above.

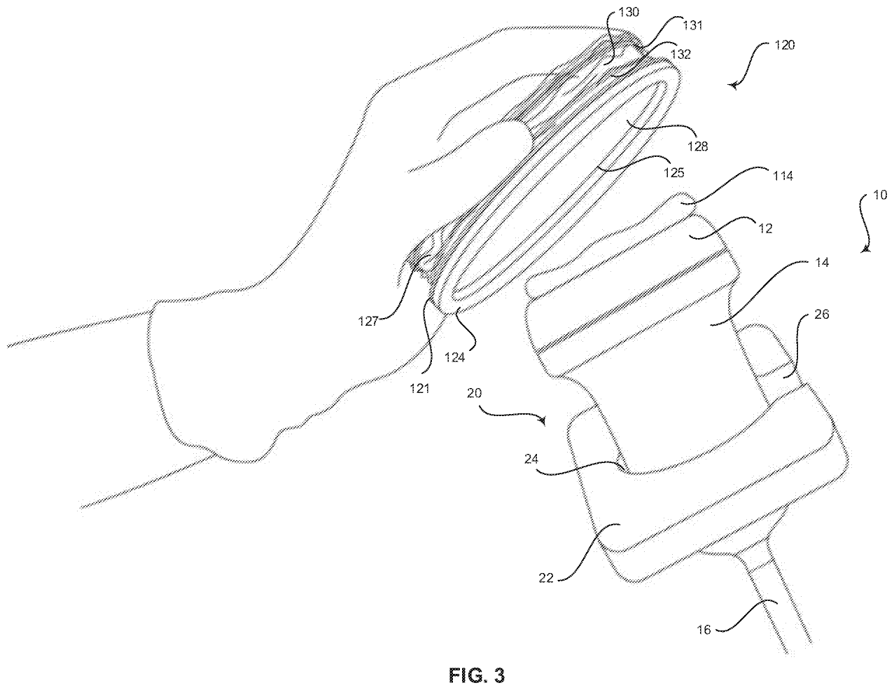

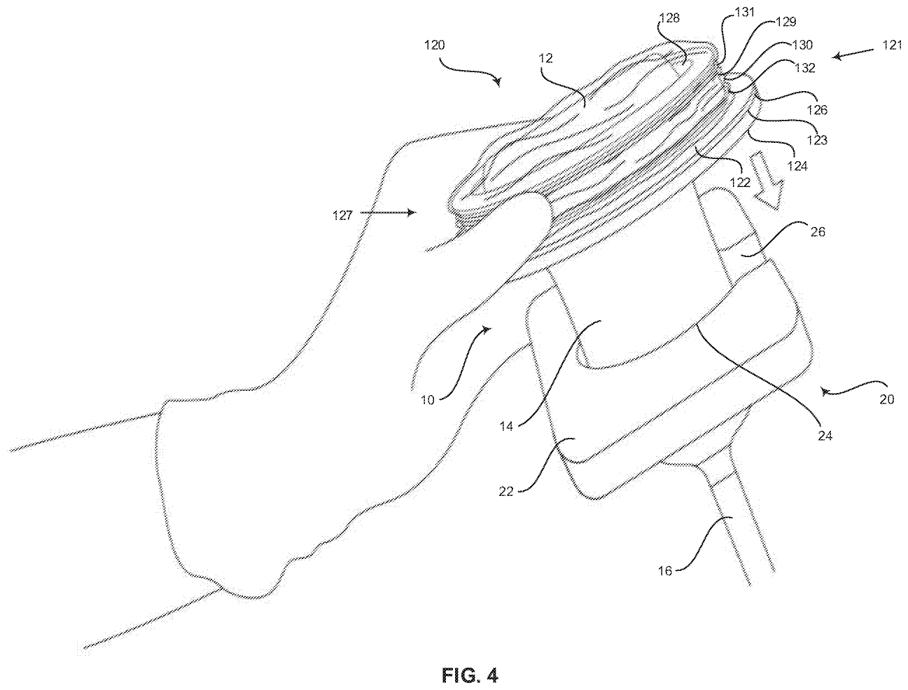

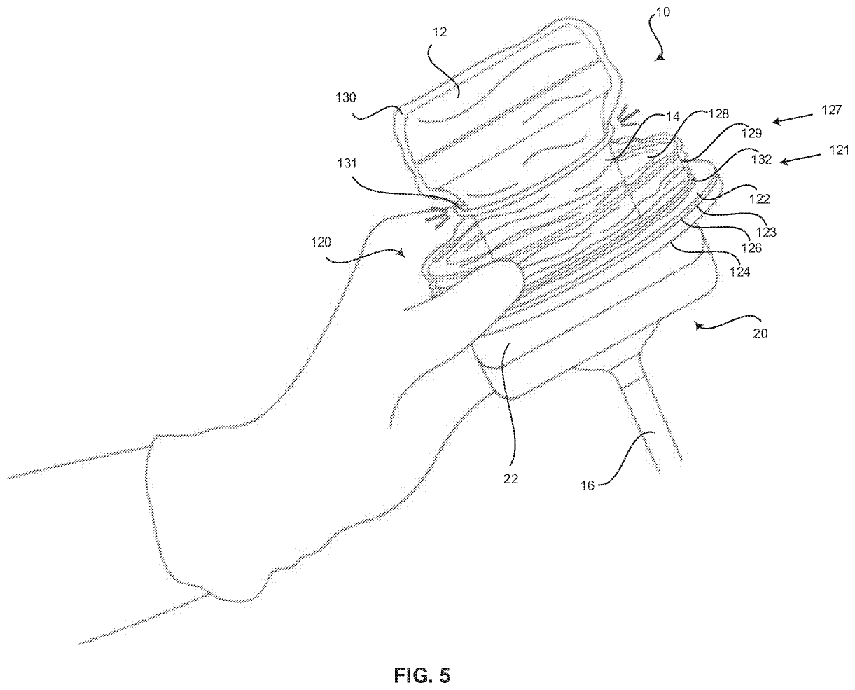

FIG. 3 illustrates an exemplary probe cover application device 120 being situated over an ultrasound probe 10 set in a probe holder 20, in accordance with an embodiment of the disclosure. FIG. 4 illustrates an exemplary probe cover application device 120 moving onto an ultrasound probe 10 set in a probe holder 20, in accordance with an embodiment of the disclosure. FIG. 5 illustrates an exemplary probe cover application device 120 being secured to the probe holder 20, in accordance with an embodiment of the disclosure. Referring to FIGS. 3-5, an ultrasound probe 10 may be set in a probe holder 20 by inserting the probe 10 through a probe insertion gap 26 in the holder 20 and setting the body 14 of the probe 10 into a probe holder opening 24 sized to hold an ultrasound probe 10. The ultrasound probe 10 may include a transducer head 12, probe body 14, and probe cord 16 connecting the ultrasound probe 10 to an ultrasound system (not shown). In various embodiments, the probe holder 20 may be part of or adjacent to a console of the ultrasound system or otherwise positioned at a location convenient to the ultrasound operator. In various embodiments, an ultrasound operator may apply gel 114 to the transducer head 12 of the ultrasound probe 10. As shown in FIGS. 3-5, an ultrasound operator grasps the probe cover application device 120, moves the probe cover application device 120 over the ultrasound probe 10 such that the probe 10 moves through the opening in the probe cover application device 120, and secures the probe cover application device 120 to the top surface 22 of the probe holder 20 with the attachment device 124 on the bottom side 123 of the base 121 of the probe cover application device 120.

The probe cover application device 120 includes a base 121 and an extension 127 that is substantially perpendicular to and extends up from the base 121. The base 121 and extension 127 (collectively also referred to as a frame) define an opening extending through the probe cover application device 120. The frame 121, 127 may be generally cylindrical, square, rectangular, triangular, hexagonal, or any suitable shape. The frame 121, 127 may be plastic or any suitable material. The base 121 of the probe cover application device 120 includes a top side 122, bottom side 123, inner edge 125, and outer edge 126. The inner edge 125 defines a portion of the opening through the probe cover application device 120. The bottom side 123 of the base 121 may include an attachment device 124, such as adhesive, detents or protrusions, hook and loop fasteners, male or female snap portions, male or female threaded portions, or the like. The top surface 22 of the probe holder 20 may be configured to receive and/or otherwise mate with the attachment device 124 of the probe cover application device 120. The extension 127 of the probe cover application device 120 includes an inside surface 128 and an outside surface 129. The inside surface 128 defines a portion of the opening through the probe cover application device 120. The outside surface 129 may be fitted with probe cover sheathing 130 that may be rolled or bunched up around the outside surface 129. The probe cover sheathing 130 may include a first closed end and a second open end. The second open end may extend over and surround the outside surface 129 of the extension 127 prior to and during deployment of the probe cover sheathing 130 onto an ultrasound probe 10. The first closed end of the probe cover sheathing 130 may extend over the opening in the extension 127 of the frame 121, 127 and may cover the transducer head 12 of the ultrasound probe 10 as the ultrasound probe is inserted through the opening in the frame 121, 127 causing the probe cover sheathing 130 to unravel, unroll, or otherwise deploy onto the ultrasound probe 10.

In various embodiments, the probe cover application device 120 may include one or more securement bands 131, 132 positioned over the probe cover sheathing 130. As the probe cover sheathing 130 is pulled off of the extension 127 of the frame 121, 127 by the ultrasound probe 10 moving through the opening in the frame 121, 127, the securement band(s) 131, 132 may also be pulled off of the extension 127 and snap onto the ultrasound probe 10 to secure the probe cover sheathing 130 between the probe 10 and the band(s) 131, 132. For example, as shown in FIG. 5, as the probe cover sheathing 130 is pulled off of the extension 127 of the frame and onto the ultrasound probe 10 passing through the opening in the frame 121, 127, a first securement band 131 may be deployed from the extension 127 to secure the probe cover sheathing 130 to the probe body 14.

The probe cover application device 120 illustrated in FIGS. 3-5 shares various characteristics with the probe cover application device 120 illustrated in FIGS. 1-2 as described above.

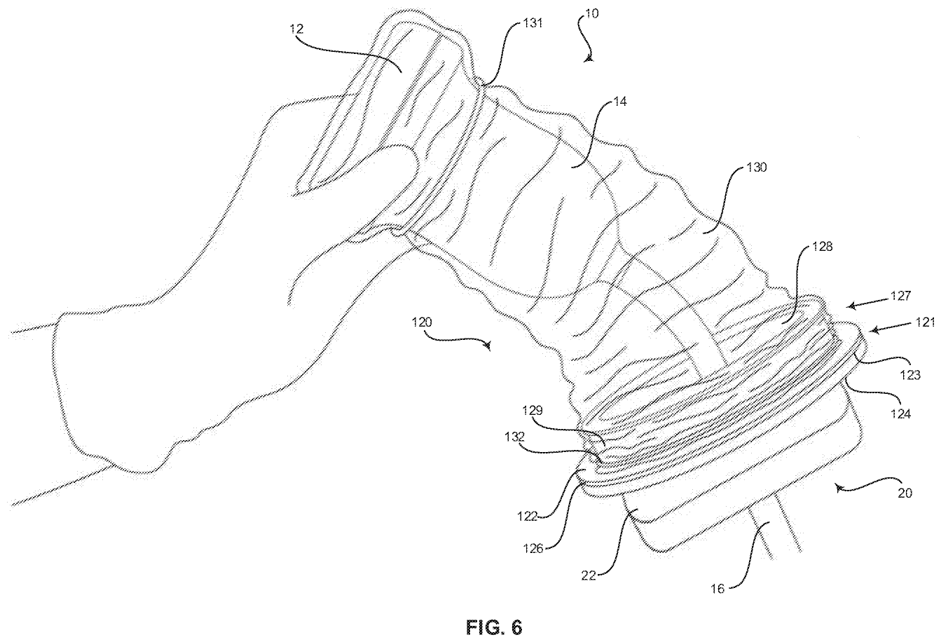

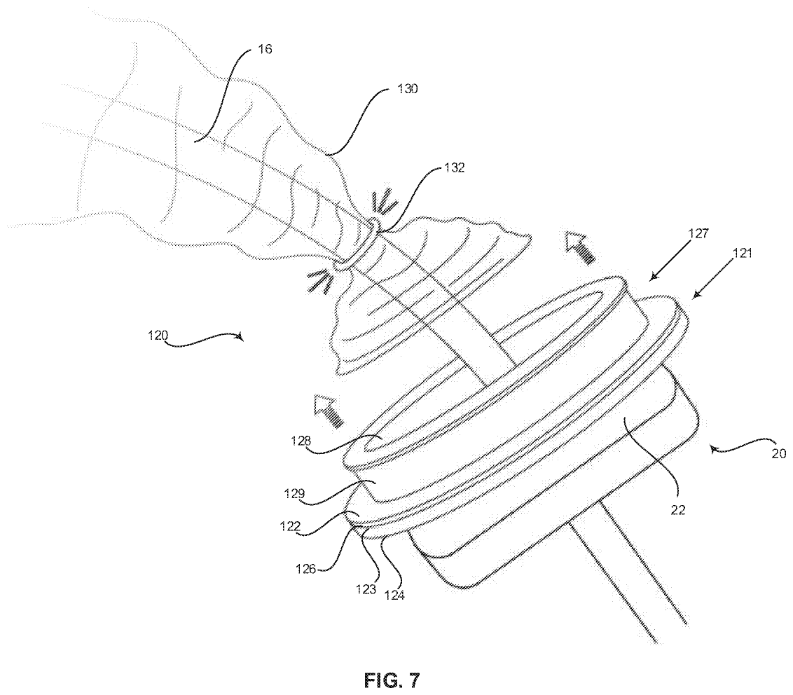

FIG. 6 illustrates exemplary probe cover sheathing 130 of an exemplary probe cover application device 120 being applied to an ultrasound probe 10, in accordance with an embodiment of the disclosure. FIG. 7 illustrates exemplary probe cover sheathing 130 of an exemplary probe cover application device 120 being secured to the ultrasound probe cord 16, in accordance with an embodiment of the disclosure. Referring to FIGS. 6-7, an ultrasound operator may grasp the portion of the ultrasound probe 10 covered by the probe cover sheathing 130 and pull the probe 10 from the probe holder 20 and through the frame 121, 127 of the probe cover application device 120 to continue unraveling the probe cover sheathing 130 onto the probe 10 after the frame 121, 127 of the probe cover application device 120 is secured to the probe holder 20 as described above with reference to FIGS. 3-5. The ultrasound probe 10 includes a transducer head 12, probe body 14, and probe cord 16 that are being covered by the probe cover application device 120. The probe cover application device 120 is secured to a probe holder 20 having a top surface 22, probe holder opening 24, and probe insertion gap 26. The ultrasound probe 10 is provided in the probe holder opening 24. The probe cover application device 120 includes a base 121 and an extension 127. The base 121 includes a top side 122, bottom side 123, inner edge 125, and outer edge 126. The probe cover application device 120 includes an attachment device 124 that can be integrated with or attached to the base 121. The attachment device 124 may be adhesive, hook and loop fasteners, snaps, detents/protrusions, threaded screws/holes, and/or any suitable attachment device. The attachment device 124 of the probe cover application device 120 is configured to mate with the probe holder 20. The extension 127 of the probe cover application device 120 is integrated with or fixedly attached to the base 121 and extends from the top side 122 of the base. The extension 127 may be substantially perpendicular to the base 121. The extension 127 includes an inside surface 128 and outside surface 129. The inside surface 128 of the extension 127 and the inner edge 125 of the base 121 form an opening that extends through the frame 121, 127.

The probe cover application device 120 includes probe cover sheathing 130 having a first closed end and a second open end. The open end of the probe cover sheathing 130 is bunched or rolled up and positioned to surround the outside surface 129 of the extension such that an inside of the probe cover sheathing 130 is accessible through the opening in the frame 121, 127. The probe cover sheathing 130 unrolls, unravels, and/or is otherwise deployed by the ultrasound probe moving through the opening in the frame 121, 127 and continuing to be pulled away from the frame 121, 127 attached to the probe holder 20. For example, an ultrasound probe 10 held in the probe holder 20 contacts the inside surface of the probe cover sheathing 130 and begins to pull the probe cover sheathing 130 off of the outside surface 129 of the extension 127. The ultrasound operator grasps the outside surface of the probe cover sheathing 130 and the ultrasound probe 10 contained therein and pulls the ultrasound probe 10 out of and away from the probe holder 20 causing the probe cover sheathing 130 to continue unraveling, unrolling, or otherwise deploying off of the outside surface 129 of the extension 127 of the frame 121, 127. In various embodiments, the probe cover sheathing 130 may be completely deployed off of the frame 121, 127 to cover the transducer head 12, probe body 14, and at least a portion of probe cord 16.

The probe cover application device 120 may include one or more securement bands 131, 132 configured to secure the probe cover sheathing 130 onto the ultrasound probe 10. For example, the securement band(s) 131, 132 may be positioned at various positions of the probe cover sheathing 130 bunched or rolled up on the outside surface 129 of the extension 127. The securement band(s) 131, 132 may be deployed from the outside surface 129 of the extension 127 with the probe sheathing 130 as the ultrasound probe moves through the opening in the frame 121, 127 thereby pulling the sheathing 130 and band(s) 131, 132 off of the extension 127. In an exemplary embodiment, a first securement band 131 may be positioned near the first, closed end of the probe cover sheathing 130 such that the first securement band 131 deploys to hold the probe cover sheathing 130 to the transducer head 12 or probe body 14 of the ultrasound probe 10 as shown in FIGS. 5-6. In a representative embodiment, a second securement band 131 may be positioned near the second, open end of the probe cover sheathing 130 such that the second securement band 132 deploys to hold the probe cover sheathing 130 to the probe cord 16 of the ultrasound probe 10 as shown in FIG. 7.

The probe cover application device 120 illustrated in FIGS. 6-7 shares various characteristics with the probe cover application device 120 illustrated in FIGS. 1-5 as described above. The ultrasound probe 10 and probe holder 20 illustrated in FIGS. 6-7 share various characteristics with the ultrasound probe 10 and probe holder 20 illustrated in FIGS. 3-5 as described above.

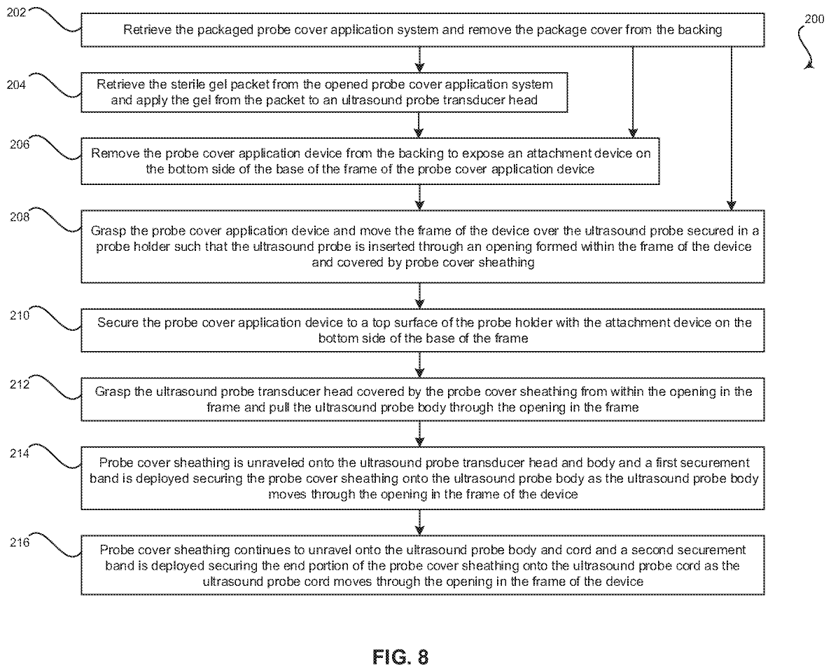

FIG. 8 is a flow chart 200 illustrating exemplary steps 202-216 that may be utilized for covering an ultrasound probe 10 with an exemplary probe cover application device 120, in accordance with an embodiment of the disclosure. Referring to FIG. 8, there is shown a flow chart 200 comprising exemplary steps 202 through 216. Certain embodiments of the present disclosure may omit one or more of the steps, and/or perform the steps in a different order than the order listed, and/or combine certain of the steps discussed below. For example, some steps may not be performed in certain embodiments of the present disclosure. As a further example, certain steps may be performed in a different temporal order, including simultaneously, than listed below.

At step 202, a packaged probe cover application system 100 is retrieved and the package cover 102 is removed from the backing 104. For example, a packaged probe cover application system 100 may include a probe cover application device 120 and accessories 110 disposed in packaging. The accessories may include, for example, tissue paper 116 and a sterile gel packet 112 configured to hold gel 114. The packaging may include, for example, a package cover 102 detachably coupled to a backing 104 that provides a sterile interior compartment for storing the probe cover application device 120 and accessories 110 prior to use. The package cover 102 may be removably attached to the backing 104 by adhesive, perforations, or any suitable coupling mechanism.

At step 204, if the accessories include a sterile gel packet 112 and the ultrasound operator desires to use gel 114, the sterile gel packet 112 may be retrieved and opened so that the gel 114 from the packet 112 may be applied to a transducer head 12 of an ultrasound probe 10. Alternatively, if a sterile gel packet 112 is not included in the packaging 102, 104, the ultrasound operator is using gel from a different source, or the ultrasound operator does not want to use gel, step 204 may be omitted and the method may continue at step 206.

At step 206, the probe cover application device 120 may be removed from the backing 104 to expose an attachment device 124 on a bottom side 123 of a base 121 of a frame 121, 127 of the probe cover application device 120. For example, the backing 104 of the packaging 102, 104 or a separate and distinct backing may cover the attachment device 124 of the probe cover application device 120. The attachment device 124 may be adhesive, hook and loop fasteners, detents/protrusions, snaps, threaded screws/holes, or any suitable attachment device. As an example, an adhesive attachment device 124 may be protected prior to use by the backing 104. The removal of the backing 104 may provide access to the adhesive attachment device 124. In exemplary embodiments, the attachment device 124 may be integrated with and/or attached to the bottom side 123 of the base. The attachment device 124 may be configured to mate with a corresponding portion of a top surface 22 of a probe holder 20. In various embodiments, backing 104 may not be attached to the attachment device 124 and step 206 may be omitted. In such embodiments, the method may proceed to step 208.

At step 208, the ultrasound operator may grasp the probe cover application device 120 and move the frame 121, 127 of the device 120 over the ultrasound probe 10 secured in a probe holder 20 such that the ultrasound probe 10 is inserted through an opening formed within the frame 121, 127 of the device 120 and covered by probe cover sheathing 130. For example, the frame 121, 127 of the probe cover application device 120 may include an inner edge 125 and inside surface 128 that defines an opening through the center of the probe cover application device 120. The probe cover sheathing 130 wraps around the outside surface 129 of the extension portion 127 of the frame 127 and covers the top of the opening. The ultrasound probe 10 held in the probe holder 20 is inserted into the opening at the bottom of the frame 121, 127 and is moved through the opening toward the top of the frame 121, 127 by moving the probe cover application device 120 over the ultrasound probe 10.

At step 210, the probe cover application device 120 may be secured to a top surface 22 of the probe holder 20 with the attachment device 124 on the bottom side 123 of the base 121 of the frame 121, 127. For example, once the probe cover application device 120 is moved over the ultrasound probe 10 such that the probe 10 extends into and through the opening in the frame 121, 127 of the probe application device 120, the bottom side 123 of the base 121 of the frame 121, 127 reaches a top surface 22 of the probe holder 20. The top surface 22 of the probe holder 20 may be configured to receive the attachment device 124 of the probe cover application device 120. As an example, the top surface 22 may: (1) be a flat surface for receiving an adhesive attachment device 124, (2) include one or more strips of hook and loop fastener configured to receive a corresponding hook and loop fastener attachment device 124, (3) include detents or protrusions configured to receive a corresponding protrusion or detent attachment device 124, (4) include male or female snaps configured to receive a corresponding female or male snap attachment device 124, and/or (5) include threaded screws or holes configured to receive a corresponding threaded hole or screw attachment device 124, among other things. In various embodiments, only one hand of the ultrasound operator may be used to secure the probe cover application device 120 to the probe holder 20.

At step 212, the ultrasound operator may grasp the ultrasound probe transducer head 12 covered by the probe cover sheathing 130 from within the opening in the frame 121, 127 and pull the ultrasound probe body 14 through the opening in the frame 121, 127. For example, with the probe cover application device 120 secured to the probe holder 20, the ultrasound operator is free to continue using one hand to apply the probe cover sheathing 130 to the ultrasound probe 10. The sterile ultrasound operator may grasp the ultrasound probe 10 while maintaining a sterile environment because the probe 10 is covered by the probe cover sheathing 130. The ultrasound operator may then pull the ultrasound probe 10, and the probe cover sheathing 130 covering the ultrasound probe 10, away from the frame 121, 127 mounted to the probe holder 20.

At step 214, the probe cover sheathing 130 is unraveled onto the ultrasound probe transducer head 12 and body 14 and a first securement band 131 is deployed to secure the probe cover sheathing 130 onto the ultrasound probe body 14 as the ultrasound probe body 14 moves through the opening in the frame 121, 127 of the device 120. For example, as the ultrasound probe 10 moves through the opening in the frame 121, 127 of the probe cover application device 120, the probe 10 will contact and apply a force to the probe cover sheathing 130 that extends and covers the opening in the extension 127 of the frame 121, 127. As the ultrasound probe 10 applies the force to the probe cover sheathing 130, the probe cover sheathing bunched up or rolled onto the outside surface 129 of the extension 127 unravels onto the ultrasound probe 10. The first securement band 131 is also deployed from the outside surface 129 of the extension 127 to secure the probe cover sheathing 130 against the transducer head 12 or probe body 14 of the ultrasound probe 10. In various embodiments, the probe cover sheathing 130 and the deployment of the first securement band 131 may occur simultaneously with steps 208-210 as shown in FIG. 5. Specifically, as the frame 121, 127 is moved over the ultrasound probe 10 so that the ultrasound probe 10 is inserted into the opening in the frame 121, 127 at step 208 until the bottom side 123 of the base 121 of the frame contacts and mates with the top surface 22 of the probe holder 20 at step 210, the ultrasound probe 10 may begin applying a force against the inside surface of the probe cover sheathing 130 that begins to unravel the probe cover sheathing 130 from the outside surface 129 of the extension 127. The probe cover sheathing 130 may then continue to unravel onto the transducer head 12 and/or body 14 at step 214 as the ultrasound probe 10 is grasped and pulled as described at step 212.

At step 216, the probe cover sheathing continues to unravel onto the ultrasound probe body 14 and cord 16 and a second securement band 132 is deployed securing the probe cover sheathing 130 onto the ultrasound probe cord 16 as the ultrasound probe cord 16 moves through the opening in the frame 121, 127 of the device 120. For example, as the ultrasound probe 10 continues to be pulled through the opening in the frame 121, 127 of the probe cover application device 120, the probe 10 will continue applying the force to the probe cover sheathing 130 and the probe cover sheathing bunched up or rolled onto the outside surface 129 of the extension 127 continues to unravel onto the ultrasound probe 10 until all of the probe cover sheathing 130 is unraveled and pulled off of the outside surface 129 of the extension 127. The second securement band 132 is also deployed from the outside surface 129 of the extension 127 to secure an end portion of the probe cover sheathing 130 against the probe cord 16 of the ultrasound probe 10. In various embodiments, additional securement bands may be deployed between the first securement band 131 and the second securement band 132 to provide additional securement of the probe cover sheathing 130 against the ultrasound probe 10.

As described above, the probe cover application device 120 allows an ultrasound operator or other user to cover an ultrasound probe 10 with probe cover sheathing 130 using only one hand and while maintaining a sterile environment (i.e., without the ultrasound probe 10 contaminating the ultrasound operator or the outside surface of the probe cover sheathing 130).

Aspects of the present disclosure provide a system and method 200 for covering an ultrasound probe 10 by a probe cover application device 120. In accordance with various embodiments, a system may comprise a frame 121, 127, an attachment device 124, and probe cover sheathing 130. The frame 121, 127 may comprise a base 121 and an extension 127. The base 121 may comprise a top side 122, a bottom side 123, an inner edge 125, and an outer edge 126. The extension 127 may extend from the top side 122 of the base 121 and substantially perpendicular to the base 121. The extension 127 may comprise an inside surface 128 and an outside surface 129. The inner edge of the base 121 and the inside surface 128 of the extension 127 may form an opening through the frame 121, 127. The attachment device 124 may be mounted to or integrated with the bottom side 123 of the base 121 of the frame 121, 127. The probe cover sheathing 130 may have a first closed end, a second open end, an inside surface, and an outside surface. The probe cover sheathing 130 may be at least one of bunched up or rolled up and positioned against and surrounding the outside surface 129 of the extension 127. The first closed end of the probe cover sheathing 130 may extend over the opening in the extension 127.

In an exemplary embodiment, the system may comprise a probe holder 20 and an ultrasound probe 10. The probe holder 20 may comprise a probe holder opening 24 and a top surface 22 at least partially surrounding the probe holder opening 24. The ultrasound probe may comprise a transducer head 12, a probe body 14, and a probe cord 16. The probe holder opening 24 of the probe holder 20 may be configured to hold the ultrasound probe 10. In a representative embodiment, the attachment device 124 may be configured to mate with the top surface 22 of the probe holder 20. In various embodiments, the attachment device 124 may be an adhesive.

In certain embodiments, the frame 121, 127 may be configured to be positioned over the ultrasound probe 10 held in the probe holder 20 and mounted to the top surface 22 of the probe holder 20 by the attachment device 124 such that at least a portion of the ultrasound probe 10 extends through the opening in the frame 121, 127. In an exemplary embodiment, the ultrasound probe 10 may be configured to move through the opening of the frame 121, 127. The movement of the ultrasound probe 10 may apply a force to the inside surface at the first closed end of the probe cover sheathing 130. The force applied to the inside surface at the first closed end of the probe cover sheathing 130 may at least one of unravel or unroll the probe cover sheathing 130 onto the ultrasound probe 10. In a representative embodiment, the ultrasound probe 10 may be at least partially through the opening of the frame 121, 127 and may be at least partially covered by the probe cover sheathing 130. The ultrasound probe 10 at least partially covered by the probe cover sheathing 130 may be configured to be pulled with the probe cover sheathing 130 to move through the opening of the frame 121, 127 and away from the frame 121, 127. The pulling of the ultrasound probe 10 with the probe sheathing 130 may apply a force to the probe cover sheathing 130. The force applied to the probe cover sheathing 130 may at least one of unravel or unroll the probe cover sheathing 130 onto the ultrasound probe 10.

In various embodiments, the system may comprise a securement band 131 positioned near the first closed end of the probe cover sheathing 130, over and against the probe cover sheathing 130, and surrounding the outside surface 129 of the extension 127. The securement band 131 may be configured to deploy from the outside surface 129 of the extension 127 with the probe cover sheathing 130 to secure the probe cover sheathing 130 to at least one of the transducer head 12 and the probe body 14 of the ultrasound probe 10 in response to the force applied to the inside surface at the first closed end of the probe cover sheathing 130 that at least one of unravels or unrolls the probe cover sheathing 130. In certain embodiments, a securement band 132 may be positioned near the second open end of the probe cover sheathing 130, over and against the probe cover sheathing 130, and surrounding the outside surface 129 of the extension 127. The securement band 132 may be configured to deploy from the outside surface 129 of the extension 127 with the probe cover sheathing 130 to secure the probe cover sheathing 130 to the probe cord 16 of the ultrasound probe 10 in response to the force applied to the probe cover sheathing 130 that at least one of unravels or unrolls the probe cover sheathing 130.

In a representative embodiment, a securement band 131 may be positioned near the first closed end of the probe cover sheathing 130, over and against the probe cover sheathing 130, and surrounding the outside surface 129 of the extension 127. In an exemplary embodiment, a securement band 132 may be positioned near the second open end of the probe cover sheathing 130, over and against the probe cover sheathing 130, and surrounding the outside surface 129 of the extension 127. In certain embodiments, the attachment device 124 may be an adhesive. In various embodiments, the system may comprise a backing 104 removably coupled to the adhesive.

In accordance with various embodiments, a method 200 may comprise moving 208 a frame 121, 127 of a probe cover application device 120 over an ultrasound probe 10 secured in a probe holder 20 such that the ultrasound probe 10 is inserted through an opening formed within the frame 121, 127 and covered by probe cover sheathing 130 of the probe cover application device 120. The frame 121, 127 may comprise a base 121 and an extension 127. The base 121 may comprise a top side 122, a bottom side 123, an inner edge 125, and an outer edge 126. The extension 127 may extend from the top side 122 of the base and substantially perpendicular to the base 121. The extension 127 may comprise an inside surface 128 and an outside surface 129. The inner edge 125 of the base 121 and the inside surface 128 of the extension 127 may form the opening through the frame 121, 127. The probe cover sheathing 130 may include a first closed end, a second open end, an inside surface, and an outside surface. The probe cover sheathing 130 may be at least one of bunched up or rolled up and positioned against and surrounding the outside surface 129 of the extension 127. The first closed end of the probe cover sheathing 130 may extend over the opening in the extension 121, 127. The method 200 may comprise securing 210 the frame 121, 127 to the probe holder 20 by an attachment device 124 mounted to or integrated with the bottom side 123 of the base 121 of the frame 121, 127. The method 200 may comprise pulling 212 the ultrasound probe 10 and the probe cover sheathing 130 away from the frame 121, 127. The method 200 may comprise at least one of unravelling or unrolling 214, 216 the probe cover sheathing 130 from the outside surface 129 of the extension 127 onto the ultrasound probe 10 as one or both of the ultrasound probe 10 is inserted through the opening formed within the frame 121, 127 and the ultrasound probe 10 and the probe cover sheathing 130 is pulled away from the frame 121, 127.

In an exemplary embodiment, the method 200 may comprise deploying 214 a securement band 131 from the outside surface 129 of the extension 127 with the probe cover sheathing 130 to secure the probe cover sheathing 130 to at least one of a transducer head 12 and a probe body 14 of the ultrasound probe 10 as one or both of the ultrasound probe 10 is inserted through the opening formed within the frame 121, 127 and the ultrasound probe 10 and the probe cover sheathing 130 is pulled away from the frame 121, 127. The securement band 131 may be positioned near the first closed end of the probe cover sheathing 130, over and against the probe cover sheathing 130, and surrounding the outside surface 129 of the extension 127 prior to deployment.

In a representative embodiment, the method 200 may comprise deploying 216 a securement band 132 from the outside surface 129 of the extension 127 with the probe cover sheathing 130 to secure the probe cover sheathing 130 to a probe cord 16 of the ultrasound probe 10 as one or both of the ultrasound probe 10 is inserted through the opening formed within the frame 121, 127 and the ultrasound probe 10 and the probe cover sheathing 130 is pulled away from the frame 121, 127. The securement band 132 may be positioned near the second open end of the probe cover sheathing 130, over and against the probe cover sheathing 130, and surrounding the outside surface 129 of the extension 127 prior to deployment.

In certain embodiments, the method 200 may comprise removing 202 the probe cover application device 120 from packaging 102, 104 by detaching a package cover 102 of the packaging 102, 104 from backing 104 of the packaging 102, 104. In various embodiments, the method 200 may comprise removing 206 the probe cover application device 120 from the backing 104 of the packaging 102, 104 to expose the attachment device 124 on the bottom side 123 of the base 121 of the frame 121, 127. In an exemplary embodiment, the attachment device 124 may be adhesive and the backing 104 may be coupled to the adhesive prior to removal. In a representative embodiment, the method 200 may comprise removing 204 accessories 110 from the packaging 102, 104. The accessories 110 may comprise tissue paper 116 and a sterile gel packet 112 comprising ultrasound gel 114.

As used herein, an element or step recited in the singular and proceeded with the word “a” or “an” should be understood as not excluding plural of said elements or steps, unless such exclusion is explicitly stated. Furthermore, references to “an embodiment,” “one embodiment,” “a representative embodiment,” “an exemplary embodiment,” “various embodiments,” “certain embodiments,” and the like are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional elements not having that property.

As utilized herein, “and/or” means any one or more of the items in the list joined by “and/or”. As an example, “x and/or y” means any element of the three-element set {(x), (y), (x, y)}. As another example, “x, y, and/or z” means any element of the seven-element set {(x), (y), (z), (x, y), (x, z), (y, z), (x, y, z)}. As utilized herein, the term “exemplary” means serving as a non-limiting example, instance, or illustration. As utilized herein, the terms “e.g.,” and “for example” set off lists of one or more non-limiting examples, instances, or illustrations. As utilized herein, a structure is “configured” and/or “operable” to perform a function whenever the structure comprises the necessary structural elements to perform the function (i.e., is designed to perform the function), regardless of whether performance of the function is disabled, or not enabled, by some user-configurable setting.

While the present disclosure has been described with reference to certain embodiments, it will be understood by those skilled in the art that various changes may be made and equivalents may be substituted without departing from the scope of the present disclosure. In addition, many modifications may be made to adapt a particular situation or material to the teachings of the present disclosure without departing from its scope. Therefore, it is intended that the present disclosure not be limited to the particular embodiment disclosed, but that the present disclosure will include all embodiments falling within the scope of the appended claims.

What is claimed is:

1. A system, comprising:
a frame comprising:
a base having a top side, a bottom side, an inner edge, and an outer edge, and
an extension extending from the top side of the base and substantially perpendicular to the base, the extension comprising an inside surface and an outside surface,
wherein the inner edge of the base and the inside surface of the extension form an opening through the frame;
an attachment device mounted to or integrated with the bottom side of the base of the frame; and
probe cover sheathing having a first closed end, a second open end, an inside surface, and an outside surface, the probe cover sheathing at least one of bunched up or rolled up and positioned against and surrounding the outside surface of the extension, the first closed end of the probe cover sheathing extending over the opening in the extension.

2. The system of claim 1, comprising:
a probe holder comprising a probe holder opening and a top surface at least partially surrounding the probe holder opening; and
an ultrasound probe comprising a transducer head, a probe body, and a probe cord,
wherein the probe holder opening of the probe holder is configured to hold the ultrasound probe.

3. The system of claim 2, wherein the attachment device is configured to mate with the top surface of the probe holder.

4. The system of claim 3, wherein the attachment device is an adhesive.

5. The system of claim 2, wherein the frame is configured to be positioned over the ultrasound probe held in the probe holder and mounted to the top surface of the probe holder by the attachment device such that at least a portion of the ultrasound probe extends through the opening in the frame.

6. The system of claim 5, wherein:
the ultrasound probe is configured to move through the opening of the frame,
the movement of the ultrasound probe applies a force to the inside surface at the first closed end of the probe cover sheathing, and
the force applied to the inside surface at the first closed end of the probe cover sheathing at least one of unravels or unrolls the probe cover sheathing onto the ultrasound probe.

7. The system of claim 5, wherein:
the ultrasound probe is at least partially through the opening of the frame and at least partially covered by the probe cover sheathing,
the ultrasound probe at least partially covered by the probe cover sheathing is configured to be pulled with the probe cover sheathing to move through the opening of the frame and away from the frame,
the pulling of the ultrasound probe with the probe sheathing applies a force to the probe cover sheathing, and
the force applied to the probe cover sheathing at least one of unravels or unrolls the probe cover sheathing onto the ultrasound probe.

8. The system of claim 6, comprising a securement band positioned near the first closed end of the probe cover sheathing, over and against the probe cover sheathing, and surrounding the outside surface of the extension, wherein the securement band is configured to deploy from the outside surface of the extension with the probe cover sheathing to secure the probe cover sheathing to at least one of the transducer head and the probe body of the ultrasound probe in response to the force applied to the inside surface at the first closed end of the probe cover sheathing that at least one of unravels or unrolls the probe cover sheathing.

9. The system of claim 7, comprising a securement band positioned near the second open end of the probe cover sheathing, over and against the probe cover sheathing, and surrounding the outside surface of the extension, wherein the securement band is configured to deploy from the outside surface of the extension with the probe cover sheathing to secure the probe cover sheathing to the probe cord of the ultrasound probe in response to the force applied to the probe cover sheathing that at least one of unravels or unrolls the probe cover sheathing.

10. The system of claim 1, comprising a securement band positioned near the first closed end of the probe cover sheathing, over and against the probe cover sheathing, and surrounding the outside surface of the extension.

11. The system of claim 1, comprising a securement band positioned near the second open end of the probe cover sheathing, over and against the probe cover sheathing, and surrounding the outside surface of the extension.

12. The system of claim 1, wherein the attachment device is an adhesive.

13. The system of claim 12, comprising a backing removably coupled to the adhesive.

14. A method, comprising:
moving a frame of a probe cover application device over an ultrasound probe secured in a probe holder such that the ultrasound probe is inserted through an opening formed within the frame and covered by probe cover sheathing of the probe cover application device, wherein:
the frame comprises a base and an extension, the base having a top side, a bottom side, an inner edge, and an outer edge, the extension extending from the top side of the base and substantially perpendicular to the base, the extension comprising an inside surface and an outside surface,
the inner edge of the base and the inside surface of the extension forms the opening through the frame, and
the probe cover sheathing includes a first closed end, a second open end, an inside surface, and an outside surface, the probe cover sheathing at least one of bunched up or rolled up and positioned against and surrounding the outside surface of the extension, the first closed end of the probe cover sheathing extending over the opening in the extension;
securing the frame to the probe holder by an attachment device mounted to or integrated with the bottom side of the base of the frame;
pulling the ultrasound probe and the probe cover sheathing away from the frame; and
at least one of unravelling or unrolling the probe cover sheathing from the outside surface of the extension onto the ultrasound probe as one or both of the ultrasound probe is inserted through the opening formed within the frame and the ultrasound probe and the probe cover sheathing is pulled away from the frame.

15. The method of claim 11, comprising deploying a securement band from the outside surface of the extension with the probe cover sheathing to secure the probe cover sheathing to at least one of a transducer head and a probe body of the ultrasound probe as one or both of the ultrasound probe is inserted through the opening formed within the frame and the ultrasound probe and the probe cover sheathing is pulled away from the frame, wherein the securement band positioned near the first closed end of the probe cover sheathing, over and against the probe cover sheathing, and surrounding the outside surface of the extension prior to deployment.

16. The method of claim 11, comprising deploying a securement band from the outside surface of the extension with the probe cover sheathing to secure the probe cover sheathing to a probe cord of the ultrasound probe as one or both of the ultrasound probe is inserted through the opening formed within the frame and the ultrasound probe and the probe cover sheathing is pulled away from the frame, wherein the securement band is positioned near the second open end of the probe cover sheathing, over and against the probe cover sheathing, and surrounding the outside surface of the extension prior to deployment.

17. The method of claim 11, comprising removing the probe cover application device from packaging by detaching a package cover of the packaging from backing of the packaging.

18. The method of claim 17, comprising removing the probe cover application device from the backing of the packaging to expose the attachment device on the bottom side of the base of the frame.

19. The method of claim 18, wherein the attachment device is adhesive and the backing is coupled to the adhesive prior to removal.

20. The method of claim 17, comprising removing accessories from the packaging, the accessories comprising tissue paper and a sterile gel packet comprising ultrasound gel.
